(19) United States
(12) Patent Application Publication
(10) Pub. No.: US 2024/0058324 A1
(43) Pub. Date: Feb. 22, 2024

(54) COMPOSITION FOR PREVENTION AND TREATING DEMENTIA THROUGH THE COMBINATION OF PDE5 INHIBITORS AND GLUCOCORTICOID RECEPTOR ANTAGONISTS

(71) Applicant: ARIBIO CO., LTD., Seongnam-si (KR)

(72) Inventors: Jaijun CHOUNG, Seongnam-si (KR); Byungwoo Kang, Busan (KR)

(73) Assignee: ARIBIO CO., LTD., Seongnam-si (KR)

(21) Appl. No.: 18/492,734

(22) Filed: Oct. 23, 2023

Related U.S. Application Data
(63) Continuation-in-part of application No. PCT/KR2022/005818, filed on Apr. 22, 2022.

(30) Foreign Application Priority Data
Apr. 23, 2021 (KR) 10-2021-0052930

(51) Int. Cl. A61K 31/4745; A61K 31/519; A61P 25/00

(52) U.S. Cl. CPC A61K 31/4745; A61K 31/519; A61P 25/00

(57) ABSTRACT

The present invention relates to the removal of amyloid-beta by the combination of mirodenafil, a phosphodiesterase 5 inhibitors (PDE5 inhibitors), and CORT-108297, a Glucocorticoid Receptor (GR) Antagonists for treatment of Alzheimer's disease or dementia.

### CROSS-REFERENCE TO RELATED APPLICATION

This application is a continuation-in-part application of PCT/KR2022/005818, filed Apr. 22, 2022, which claims the benefit of Korean Patent Application No. 10-2021-0052930, filed Apr. 23, 2021, the contents of each of which are incorporated herein by reference.

### FIELD

The present invention relates to a composition containing a phosphodiesterase 5 (PED5) inhibitor, and a glucocorticoid receptor (GR) antagonist and a method of preventing of treating Alzheimer's disease or dementia or reduction of neuroinflammation using the same.

### BACKGROUND

Dementia is an acquired brain disease with multifaceted pathogenesis caused by various genetic and environmental risk factors, and refers to a clinical disease that suffers from multiple cognitive deficits. The most common cause of dementia is Alzheimer's disease, which mainly occurs in the elderly and is reported to account for about 60-70% of all dementia cases (Ann Neurol. 1993 May; 33(5):494-501.).

Amyloid beta protein (Aβ), which is known to be a common cause of hereditary and sporadic Alzheimer's disease, is actively being studied. Aβ is produced in small amounts throughout the human body, even in normal people. In normal people, Aβ is quickly decomposed after it is made and does not accumulate in the human body, but in patients with Alzheimer's disease, Aβ is produced abnormally in large quantity and does not decompose and accumulates in tissues, resulting in the formation of senile plaques or excessive accumulation in places such as the hippocampus and cerebral cortex, which play an important role in memory and learning. The accumulated Aβ causes an inflammatory response in the surrounding cells. As a result, nerve cells are damaged and, gradually, the neural circuitry that maintains the normal functioning of the brain is compromised. In addition, the accumulated Aβ produces a lot of free radicals that activate the signaling system that kills nerve cells.

Aβ is part of an amyloid precursor protein cleaved by β-secretase. Aβ comes in several forms, depending on the number of amino acids that make it up. In patients with Alzheimer's disease, the ratio of Aβ, which is made up of 40 or 42 amino acids, increases dramatically. When Aβ is treated with nerve cells cultured in vitro, it induces neuronal death, and there are many reports that the mechanism of cell death is similar to the type of apoptosis seen in patients with Alzheimer's disease. Damage to nerve cells by Aβ1-42 or Aβ1-43 proteins has been identified as one of the important causes of Alzheimer's disease, and Aβ25-35 is known to be an important toxic fragment of Aβ1-42 or 43 that causes damage to nerve cells.

Some of the most commonly FDA-approved drugs currently used to treat dementia include AChEI and N-Methyl-D-aspartate receptor antagonists, as well as antioxidants, nonsteroidal anti-inflammatory drugs (NSAIDs), anti-inflammatory agents, statins, and hormones. Various drugs and its formulations are used interchangeably with them. However, these drugs are only used to alleviate symptoms, delay and improve cognitive function, but no fundamental treatment for dementia has been developed.

Typical AChEIs include donepezil, rivastigmine, and galantamine, which have a symptomatic treatment effect by temporarily increasing the concentration of the neurotransmitter acetylcholine. These drugs are also prescribed for mild to moderate Alzheimer's disease, vascular dementia, Parkinson's disease dementia and stroke or subcortical ischemic vascular disease.

Currently, global pharmaceutical companies are trying to develop drugs that inhibit the action of β-secretase to block the production of Aβ at the source. In recent years, pharmaceutical companies, universities, and research institutes in the United States have been conducting many studies to isolate components that exhibit antioxidant effects from medicinal plants for the development of dementia treatments, and among them, the ingredients that have begun to be used as a treatment for dementia are Ginkgo biloba L (Ginkgo biloba) and Huperzia serrata. These are typical cases. In addition, curcuminoids isolated from Curcuma longa L. (turmeric) have also been reported to have therapeutic efficacy in dementia. Ginseng, tetrandrine, and ginkgo biloba extract (EGB761), which are known to inhibit the toxicity of amyloid metabolites, have been reported to increase neuronal survival or slow the rate of worsening of dementia symptoms in Alzheimer's patients.

On the other hand, there is no satisfactory treatment method for Alzheimer's dementia, and effective anti-dementia drugs have not yet been developed.

Degenerative neurological diseases, including dementia, are caused by the reduction or loss of nerve cell function, which causes abnormalities in a wide variety of functions, including autonomic nervous function, which is self-regulating without perceiving as well as all functions of the body that we can feel, such as motor control function, cognitive function, perceptive function, and sensory function.

Currently, the cause of dementia has not been identified, so fundamental treatment is not possible, and the five commercialized drugs can only alleviate symptoms in some diseases, and these drugs have only the effect of symptom completion that does not fundamentally change the progression of dementia.

### SUMMARY

One embodiment of the present invention provides a composition comprising a phosphodiesterase 5 inhibitor; and a glucocorticoid receptor antagonists (Glucocorticoid Receptor (GR) Antagonist) as active ingredients.

Another embodiment of the present invention provides a method for treating dementia by protecting nerve cells by reducing amyloid beta by using mirodenafil alone or in combination with CORT-108297, which is being developed as a treatment for post-traumatic stress disorder (PTDS), against amyloid beta, which is a direct cause of neuronal death.

Yet another embodiment of the present invention provides a methods for reducing neuroinflammation.

The present invention also provides a method for the prevention and treatment of Alzheimer's disease or dementia comprising:
administering a composition comprising a phosphodiesterase 5 inhibitor; and a glucocorticoid receptor antagonists (Glucocorticoid Receptor (GR) Antagonist) as active ingredients.

In an embodiment of the present invention provides a composition for preventing and treating dementia comprising a phosphodiesterase 5 inhibitor and a Glucocorticoid Receptor (GR) Antagonist as active ingredients.

In a certain embodiment of the present invention, the composition comprises the weight % of PDE-5 inhibitor and a glucocorticoid receptor antagonists (Glucocorticoid Receptor (GR) Antagonist) a glucocorticoid receptor antagonists (Glucocorticoid Receptor (GR) Antagonist) a glucocorticoid receptor antagonists (Glucocorticoid Receptor (GR) Antagonist) is from 1:0.1 to 1:10 or 50:1, 10:1, 5:1, 2:1, 1:1, 1:2, 1:5, or 1:10.

In another embodiment, the compositions of the present invention provides a synergistic effect for:
inhibition of Aβ Oligomer/Fibril formation by reduction of Aβ aggregation;
inhibition of β-Amyloidogenic Processing decreased BACE-1;
reduction of extracellular Aβ monomers, oligomers & Aβ Fibril/Plaque by increase of the cerebral blood flow;
suppression of neuronal cell death Inhibition and promotion of neurogenesis, synaptogenesis and/or angiogenesis by activation of NO/cGMP/PKG/CREB Pathway,
restoration of synaptic plasticity (synaptic Plasticity) by activation of Wint Signaling by inhibition of DKK-1, and inhibition of production of APP and reduction of AB accumulation by suppression of positive feedback loop for Aβ production, and
inhibition of formation of Aβ Fibril/plaque by removal of intracellular toxic and soluble Aβ oligomers by activation of autophagy.

In a certain embodiment, the compositions of the present invention also provide synergistic effects on inhibition of proinflammatory factors to provide reduction of neuroinflammation.

### BRIEF DESCRIPTION OF THE DRAWING

FIG. 1 and FIG. 2 are experimental results of a reduction in intracellular Aβ by co-treatment of mirodenafil (AR1001) and CORT-108297 according to an embodiment of the present invention.

FIG. 3 shows the synergistic effect on IL-1β reduction in the present invention of the combined treatment of mirodenafil and CORT-108297. Here, AR1001 refers to the mirodenafil.

FIG. 4 shows the synergistic effect on TNF-α reduction in the present invention of the combined treatment of mirodenafil and CORT-108297. Here, AR1001 refers to the mirodenafil.

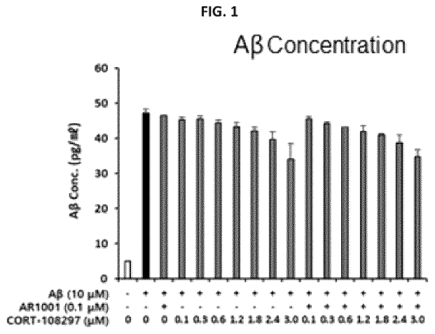

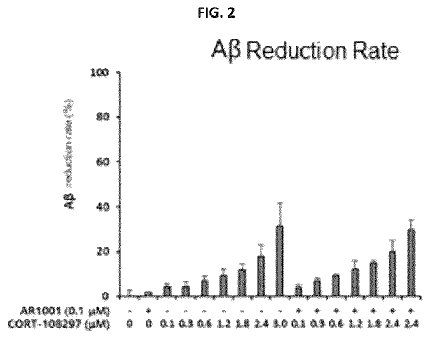

### DETAILED DESCRIPTION

The present invention provides a composition comprising a phosphodiesterase 5 (PDE-5) inhibitor; and a glucocorticoid receptor antagonists (GR antagonists) as active ingredients.

The present invention also provides a method for the prevention and treatment of Alzheimer's disease or dementia comprising:
administering a composition comprising a phosphodiesterase 5 inhibitor; and a glucocorticoid receptor antagonists (GR antagonist) as active ingredients.

In an embodiment of the present invention provides a composition for preventing and treating dementia comprising a phosphodiesterase 5 inhibitor and an GR antagonist as active ingredients.

In a certain embodiment of the present invention, the composition comprises the weight % of PDE-5 inhibitor and a glucocorticoid receptor antagonists (GR Antagonist) is 1:1-1:24 or 1:20, 1:15, 1:10, 1:8, 1:5, 1:4, 1:3, 1:2, or 1:1.

In another embodiment, the compositions of the present invention provides a synergistic effect for:
inhibition of Aβ Oligomer/Fibril formation by reduction of Aβ aggregation;
inhibition of β-Amyloidogenic Processing decreased BACE-1;
reduction of extracellular Aβ monomers, oligomers & Aβ Fibril/Plaque by increase of the cerebral blood flow;
suppression of neuronal cell death Inhibition and promotion of neurogenesis, synaptogenesis and/or angiogenesis by activation of NO/cGMP/PKG/CREB Pathway,
restoration of synaptic plasticity (synaptic Plasticity) by activation of Wint Signaling by inhibition of DKK-1, and inhibition of production of APP and reduction of Aβ accumulation by suppression of positive feedback loop for Aβ production, and
inhibition of formation of Aβ Fibril/plaque by removal of intracellular toxic and soluble Aβ oligomers by activation of autophagy.

The present invention relates to the effect of inhibiting dementia through amyloid beta reduction by combining phosphodiesterase 5 inhibitor and glucocorticoid receptor antagonist (Glucocorticoid Receptor (GR) Antagonist).

The present invention provides a composition and a method for treating a neurodegenerative disease by reducing the neuroinflammation especially in CNS system and/or by reducing the expression of a toxic protein such as beta-amyloid (Aβ),
wherein,
the composition comprising a PDE-5 inhibitor and a GR antagonist,
   wherein,
   the PDE-5 is from among mirodenafil, sildenafil, vardenafil, tadalafil, udenafil, dasantafil, avanafil; and pharmaceutically acceptable salts, solvates, hydrates, or a mixture thereof;
   the GR antagonist is selected from among CORT-108297, prednisolone, dexamethasone, mifepristone, ciclesonide, budesonide, cortisone, flunisolide; and at least one species selected from a group consisting of pharmaceutically acceptable salts, solvates, or hydrates thereof.

In a certain embodiment, the compositions of the present invention also provide synergistic effects on inhibition of proinflammatory factors to provide reduction of neuroinflammation.

In the present invention, dementia includes Alzheimer's dementia, AIDS-induced dementia, Lewy body dementia, frontotemporal dementia, multiple infarction dementia, semantic dementia and vascular dementia, and Huntington's disease, Parkinson's disease, and amyotrophic lateral sclerosis.

The phosphodiesterase 5 inhibitor of the present invention comprises mirodenafil, sildenafil, vardenafil, tadalafil, udenafil, dasantafil, avanafil; and at least one species selected from a group consisting of pharmaceutically acceptable salts, solvates or hydrates thereof.

The pharmaceutically acceptable salt means a formulation of a compound that does not cause serious irritation to the organism to which the compound is administered and does not impair the biological activity and physical properties of the compound. The pharmaceutically acceptable salts are prepared by conventional methods well known in the art using pharmaceutically acceptable organic and inorganic acids that are substantially non-toxic. The acids are inorganic acids such as hydrochloric acid, bromic acid, sulfuric acid, nitric acid, phosphoric acid, methanesulfonic acid, ethanesulfonic acid, sulfonic acid such as p-toluenesulfonic acid, tataric acid, formic acid, citric acid, acetic acid, trichloroacetic acid, trifluoroacetic acid, capric acid, isobutanoic acid. It includes organic acids such as malonic acid, succinic acid, phthalic acid, gluconic acid, benzoic acid, lactic acid, fumaric acid, maleic acid, salicylic acid, and the like. In addition, by reacting the compounds of the present invention with bases, ammonium salts; alkali metal salts, such as sodium or potassium salts; salts such as calcium or magnesium salts, alkali earth metal salts, etc.; Salts of organic bases such as dicyclohexylamine, N-methyl-D-glucamine, tris (hydroxymethyl) methylamine and the like; And amino acid salts such as arginine and lysine may be formed.

According to an embodiment of the present invention, the pharmaceutically acceptable salts may illustrate mirodenafil hydrochloride, sildenafil citrate, bardenafil hydrochloride, and the like.

The hydrate refers to a compound of the present invention or a salt thereof comprising a stoichiometric or non-stoichiometric amount of water bonded by a non-covalent intermolecular force.

The solvate means a compound of the present invention or a salt thereof comprising a stoichiometric or non-stoichiometric amount of solvent bonded by a non-covalent intermolecular force. Preferred solvents are volatile, non-toxic, and/or solvents suitable for administration to humans.

Glucocorticoid Receptor (GR) antagonist is selected from among CORT-108297, prednisolone, dexamethasone, mifepristone, ciclesonide, budesonide, cortisone, flunisolide; and at least one species selected from a group consisting of pharmaceutically acceptable salts, solvates or hydrates thereof.

More preferably, the phosphodiesterase 5 inhibitor of the present invention is at least one selected from the group consisting of mirodenafil and its pharmaceutically acceptable salts, solvates or hydrates, and the GR antagonist is at least one of the selected from CORT-108297 and pharmaceutically acceptable salts, solvates or hydrates thereof.

CORT-108297 of the present invention is (R)-(4a-ethoxymethyl-1-(4-fluorophenyl)-6-(4-trifluoromethyl-benzenesulfonyl)-4,4a,5,6,7,8-hexahydro-1H,1,2,6-triaza-cyclopenta[b] naphthalene, which has the following structure:

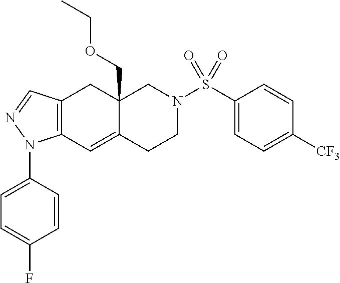 [SMILES: CCOC[C@]12Cc3cnn(-c4ccc(F)cc4)c3C=C1CCN(S(=O)(=O)c1ccc(C(F)(F)F)cc1)C2]

The pharmaceutical composition of the present invention can be administered orally or parenterally.

According to an embodiment of the present invention, the pharmaceutical composition of the present invention is orally administered to a subject, or non-orally administered to a site other than the head. That is, the compositions of the present invention may exhibit the intended effect in the present invention even when not directly administered to brain tissue, body tissues surrounding brain tissue (eg, scalp) and adjacent areas. In one particular example, the non-oral administration is subcutaneous administration, intravenous administration, abdominal infusion, transdermal administration, or intramuscular administration, and in another particular example, subcutaneous administration, intravenous administration, or intramuscular administration.

The pharmaceutically acceptable carriers contained in the pharmaceutical composition of the present invention are commonly used in the formulation of lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, Syrup, methyl cellulose, methyl hydroxybenzoate, propyl hydroxybenzoate, talc, magnesium stearate and mineral oil, but are not limited to. The pharmaceutical composition of the present invention may additionally comprise a lubricant, a humectant, a sweetener, a flavoring agent, an emulsifier, a suspension, a preservative, and the like in addition to the above components. Suitable pharmacologically acceptable carriers and formulations are described in detail in Remington's Pharmaceutical Sciences (19th ed., 1995).

The pharmaceutical composition of the present invention may be prepared in a unit dose form or in a multi-capacity container by formulation using a pharmaceutically acceptable carrier and/or excipient according to a method that can be easily performed by a person skilled in the art to which the invention belongs. In this case, the formulation may be in the form of a solution, suspension or emulsion in an oil or aqueous medium, or in the form of an X-agent, powder, granule, tablet, film or capsule, and may additionally include a dispersant or stabilizer.

### EXAMPLES

Hereinafter, the present invention will be described in more detail according to the following embodiments. However, these embodiments are only for illustrative purposes of the present invention, and the scope of the present invention is not limited by these embodiments.

### Example 1

### Cell Culture

The SH-SY5Y human neuroblastoma cell line used in the experiment was obtained from the American Type Culture Collection (ATCC; Manassas, VA, USA) and 10% fetal bovine serum (FBS; Australian Orgin, HyClone, Logan, UT, USA) and 1% penicillin/streptomycin (P/S; HyClone) was incubated in a CO2 incubator (311-TIF, Thermo Fisher Scientific Forma, MA, USA) under 37° C. and 5% CO2 conditions using DMEM/F12 Complete Medium (HyClone).

### Example 2

### Neuron-Like Differentiation of SH-SY5Y Cells Using All-Trans-Retinoic Acid (RA)

To determine the amount of change in amyloid beta, 2*?*⁵ cells were dispensed into T-25 flask.

For cell adhesion and stabilization, cells were incubated for 24 hours in DMEM/F12 Complete Medium (HyClone) containing 10% FBS (HyClone) and 1% P/S (HyClone) at 37° C. under 5% CO₂ condition in a CO₂ incubator (Thermo Fisher Scientific Forma).

After 24 hours of cell dispensing, the cell culture medium is removed for neuron-like differentiation, and replaced with DMEM/F12 differentiation media containing 1% FBS (HyClone), 1% P/S (HyClone), 10 μM all-trans-retinoic acid (RA; Sigma--Aldrich, St. Louis, MO, USA).

On the third day of differentiation, the medium was replaced with a new DMEM/F12 differentiation medium. On the sixth day of differentiation, the medium of the untreated control group was replaced with a new DMEM/F12 differentiation medium and the medium of the sample treatment group was replaced with a various conditions by adding a new DMEM/F12 differentiation medium under various conditions.

### Example 3

### Amyloid β(Aβ)1-42 Formation and Treatment

Human Aβ1-42 (Abeam, Cambridge, MA, USA) was added upto 10 μM to DMEM/F12 Complete Medium (HyClone) containing 1% FBS (HyClone) and 1% P/S (HyClone) to form Aβ1-42 oligomer and left for 3 hours in a CO₂ incubator (Thermo Fisher Scientific Forma) under 37° C. and 5% CO₂ conditions to form Aβ1-42 oligomer.

In order to confirm the Aβ 1-42 change, the existing cell culture medium was removed from the RA-differentiated SH-SY5Y neuron-like cells, replaced with DMEM/F12 Complete Medium (HyClone) containing Aβ1-42 oligomer (10 μM) and incubated for 72 hours in a CO₂ incubator (Thermo Fisher Scientific Forma) at 37° C. under 5% CO₂ conditions to induce Aβ1-42 oligomer-induced cell damage.

After 72 hours, the medium was removed and the cells were treated with of DMEM/F12 Complete Medium (HyClone) containing 10 μM of Aβ1-42 oligomer alone or in combination with mirodenafil and CORT-108297 and incubated in a CO₂ incubator (Thermo Fisher Scientific Forma) at 37° C. under 5% CO₂ for 24 hours before the next experiments.

### Example 4

### Amyloid Beta 42 Human ELISA (Enzyme-Linked Immunosorbent Assay) Measurement Results

In order to measure the amount of Aβ42 (pg/mL) in the cell, the cells were recovered and treated with the cell lysis buffer.

Thereafter, after centrifugation at 14,000 rpm 4° C. for 10 minutes, the supernatant was transferred to recover the protein. The amount of protein was quantified using the Pierce™ BCA Protein Assay Kit (Thermo Fisher Scientific). Then, the amount of Aβ42 in cells was measured using a Human Aβ42 ELISA Kit (Invitrogen).

TABLE 1

| Sample | | | | | Aβ42 Reduction rate | | | |
|---|---|---|---|---|---|---|---|---|
| | CORT- | | Aβ42 concentration | | Average | | | |
| | 108297 | Aβ | Average | Concentration | reduction | Reduction | Combination Index | |
| AR10001 | (μM) | (10 μM) | concentration | Stdev | rate | rate | Group | Value |
| − | 0 | − | 4.99 | 0.19 | − | | − | |
| − | 0 | + | 47.19 | 1.16 | 0.00 | 2.75 | − | |
| + | 0 | + | 46.45 | 0.11 | 1.77 | 0.25 | A | |
| − | 0.1 | + | 45.40 | 0.59 | 4.26 | 1.39 | B | |
| − | 0.3 | + | 45.40 | 0.95 | 4.24 | 2.24 | | |
| − | 0.6 | + | 44.29 | 1.05 | 6.88 | 2.49 | | |
| − | 1.2 | + | 43.25 | 1.26 | 9.34 | 2.98 | | |
| − | 1.8 | + | 42.14 | 1.15 | 11.97 | 2.73 | | |
| − | 2.4 | + | 39.64 | 2.19 | 17.91 | 5.18 | | |
| − | 3.0 | + | 33.97 | 4.47 | 31.34 | 10.59 | | |
| + | 0.1 | + | 45.59 | 0.66 | 3.81 | 1.56 | Combination | 0.63 |
| + | 0.3 | + | 44.22 | 0.52 | 7.06 | 1.24 | | 1.17 |
| + | 0.6 | + | 43.18 | 0.10 | 9.52 | 0.25 | | 1.10 |
| + | 1.2 | + | 41.99 | 1.57 | 12.32 | 3.72 | | 1.11 |
| + | 1.8 | + | 40.89 | 0.42 | 14.95 | 0.99 | | 1.09 |
| + | 2.4 | + | 38.75 | 2.18 | 20.00 | 5.17 | | 1.02 |
| + | 3.0 | + | 34.72 | 2.07 | 29.57 | 4.91 | | 0.89 |

In conclusion, the Aβ reduction rate was 7.06% in mirodenafil 0.1 μM and CORT-108297 0.3 μM combo treatment group; 9.52% Aβ reduction rate in mirodenafil 0.1 μM and CORT-108297 0.6 μM combo treatment group; 12.32% Aβ reduction rate in mirodenafil 0.1 μM and CORT-108297 1.2 μM combo treatment group; 14.95% Aβ reduction rate in mirodenafil 0.1 μM and CORT-108297 1.8 μM combo treatment group; 20.00% Aβ reduction rate in mirodenafil 0.1 μM and CORT-108297 2.4 μM combo treatment group; all of which were higher than the sum of A and B reduction rate from treatment with mirodenafil or CORT-108297 individually and thus, it was confirmed that the effect was more than additive and synergistic.

### Example 5

### Culture Method of IMG Cells

The IMG cells, a mouse microglia cell line used in the experiments, were cultured, and maintained in DMEM complete medium (HyClone) containing 10% fetal bovine serum (FBS; Australian Orgin, HyClone, Logan, UT, USA) and 1% penicillin/streptomycin (P/S; HyClone) at 37° C. with 5% CO₂ in a humidified CO₂ incubator (311-TIF, Thermo Fisher Scientific Forma, MA, USA). Total 2×10\hat{ }5 cells were seeded in each well of the 6-well plate and they were incubated for 24 h in the humidified CO₂ incubator as mentioned above. Further, 100 ng/ml of LPS and the drugs, AR1001 2 μM and GR modulator (CORT-108297) 6, 12, 24, 36, 48 μM were treated individually or in combination.

### Example 6

### RNA Extraction and cDNA Synthesis

The cells were scraped using a cell scraper, and 2 mL of the culture solution was collected in a 15 mL conical tube, It was centrifuged at 3,000 RPM for 5 min and the supernatant (culture solution) was discarded. The pellet was resuspended in 1 mL of Trizol and it was transferred to 1.5 mL microfuge tube. Further, 0.2 mL of chloroform was added and it was vortexed for 1 min. The microfuge tube was kept in a stand for 2 min at room temperature. After centrifugation at 12,000×g for 10 min at 4° C., the supernatant (approx 500 μL) separated in a fresh microfuge tube. Equal volume of isopropanol was added to the supernatant and mixed the solution well. It was put in the microfuge tube stand for 10 min at room temperature and thereafter, centrifuged at 12,000×g for 10 min at 4° C. The supernatant was discarded and the pellet was washed twice with 75% ethanol. The RNA pellet was dried and dissolved in 10 μL DEPC treated water. After quantification of the RNA, it was converted to cDNA following the PrimeScript™ II 1st strand cDNA Synthesis Kit (Takara) protocol.

### Example 7

### Real Time RT-qPCR of Pro-Inflammatory Cytokines

The sample cDNAs were amplified with gene specific primers and SYBR green PCR master mix (ThermoFisher) in the model Quant Studio 5 Thermal cycler (Applied biosystems). The amplification conditions were as follows—polymerase activation at 50° C. for 2 min, predenaturation preceding at 95° C. for 10 min, total 40 cycles of denaturation at 95° C. for 15 sec, annealing at 60° C. for 30 sec and extension at 72° C. for 30 sec. The specific primer sequences are mentioned in Table-1.

TABLE 2
Primer sequences for Real Time RT-PCR

| Gene name | Primer sequence |
|---|---|
| IL1β | Forward 5′-AGCTTCAGGCAGGCAGTATC-3′ |
| | Reverse 5′-AAGGTCCACGGGAAAGACAC-3′ |
| TNFα | Forward 5′-AAATGGCCTCCCTCTCATCAG-3′ |
| | Reverse 5′-GTCACTCGAATTTTGAGAAGATGATC-3′ |
| β actin | Forward 5′-CGTGCGTGACATCAAAGAGAA-3′ |
| | Reverse 5′-TGGATGCCACAGGATTCCAT-3′ |

### Example 8

### Measurement of Pro-Inflammatory Cytokines Level

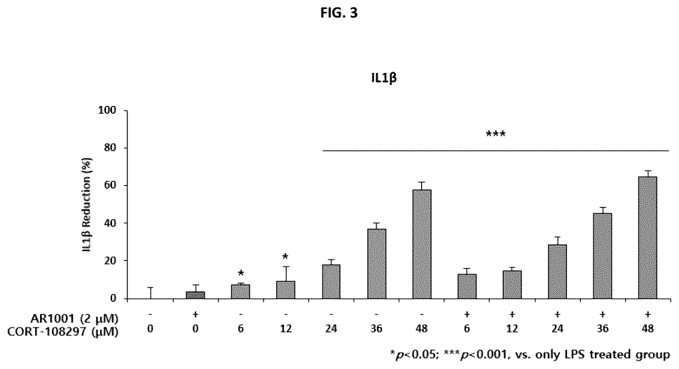

FIG. 3 shows the synergistic effect on IL-1β in the present invention of the combined treatment of mirodenafil and CORT-108297. Here, AR1001 refers to the mirodenafil.

In Table 3, the IL-1β reduction rate for the combined treatment of 2 μM of mirodenafil and 6 μM of CORT-108297 was 12.62%; for the combination of 2 μM of mirodenafil and 12 μM of CORT-108297, the reduction rate was 14.89%; for 2 μM of mirodenafil and 24 μM of CORT-108297 combination, the reduction rate was 26.04%; for 2 μM of mirodenafil and 36 μM of CORT-108297 combination, the reduction rate was 45.11%; and for 2 μM of mirodenafil and 48 μM of CORT-108297 combination, the reduction rate was 65.08%. The reduction rates of the different combinations were significantly higher than the sum of the increase rates A and B for the treatment of mirodenafil or CORT-108297 alone, which confirmed that an effect beyond the additive effect can be recognized.

TABLE 3
IL-1β reduction rate

| | | | IL1β | | |
|---|---|---|---|---|---|
| Sample | | | | Synergistic Effect | |
| AR1001 | CORT-108297 | LPS | Reduction | Evaluation | |
| (2 μM) | (μM) | (100 ng/ml) | (%) | (AB)/(A + B) | |
| − | 0 | − | — | — | |
| − | 0 | + | 0.00 | — | |
| + | 0 | + | 4.70 | A | |
| − | 6 | + | 7.02 | B1 | |
| − | 12 | + | 8.38 | B2 | |
| − | 24 | + | 17.88 | B3 | |
| − | 36 | + | 35.86 | B4 | |
| − | 48 | + | 57.37 | B5 | |
| + | 6 | + | 12.62 | AB1 | 1.17 |
| + | 12 | + | 14.89 | AB2 | 1.13 |
| + | 24 | + | 26.04 | AB3 | 1.32 |
| + | 36 | + | 45.11 | AB4 | 1.11 |
| + | 48 | + | 65.08 | AB5 | 1.05 |

A: Percentage Decrease of AR1001 treated alone; B: Percentage Decrease of CORT-108297 treated alone; A+B:
Percentage Decrease of combined treatment of AR1001 and CORT-108297
Synergistic Effect Evaluation: >1 Synergistic Effect; =1 Additive Effect; <1 Antagonistic Effect
AR1001 and CORT-108297 has synergistic effect on IL1β inhibition at the ratio of AR1001:CORT-108297=1:3, 1:6, 1:12, 1:18, 1:24

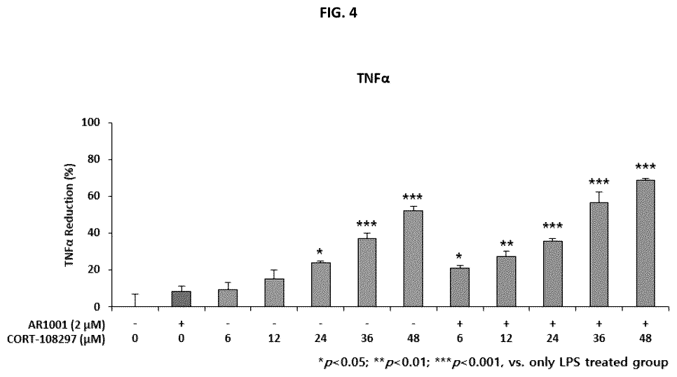

FIG. 4 shows the synergistic effect on TNF-α in the present invention of the combined treatment of mirodenafil and CORT-108297. Here, AR1001 refers to the mirodenafil.

In Table 4, the TNF-α reduction rate for a combined treatment of 2 μM of mirodenafil and 6 μM of CORT-108297 was 20.99%; for the combination of 2 μM of mirodenafil and 12 μM of CORT-108297, the reduction rate was 27.23%; for the combination of 2 μM of mirodenafil and 24 μM of CORT-108297, the reduction rate was 35.93%; for 2 μM of mirodenafil and 36 μM of CORT-108297 combination, the reduction rate was 56.64%; and for 2 μM of mirodenafil and 48 μM of CORT-108297 combination, the reduction rate was 68.70%. The reduction rates of the different combinations were significantly higher than the sum of the increase rates A and B for the treatment of mirodenafil or CORT-108297 alone, which confirmed that an effect beyond the additive effect can be recognized.

TABLE 4
TNF-α reduction rate

| | | | TNFα | | |
|---|---|---|---|---|---|
| Sample | | | | Synergistic Effect | |
| AR1001 | CORT-108297 | LPS | Reduction | Evaluation | |
| (2 μM) | (μM) | (100 ng/ml) | (%) | (AB)/(A + B) | |
| − | 0 | − | — | — | |
| − | 0 | + | 0.00 | — | |
| + | 0 | + | 8.58 | A | |
| − | 6 | + | 9.41 | B1 | |
| − | 12 | + | 15.52 | B2 | |
| − | 24 | + | 23.86 | B3 | |
| − | 36 | + | 37.30 | B4 | |
| − | 48 | + | 52.51 | B5 | |
| + | 6 | + | 20.99 | AB1 | 1.32 |
| + | 12 | + | 27.23 | AB2 | 1.17 |
| + | 24 | + | 35.93 | AB3 | 1.14 |
| + | 36 | + | 56.64 | AB4 | 1.30 |
| + | 48 | + | 68.70 | AB5 | 1.12 |

The present invention described above is only an example, and a person of ordinary skill in the art to which the present invention belongs will be well aware that various modifications and uniform other embodiments are possible therefrom. Therefore, it may be well understood that the present invention is not limited to the forms referred to in the above detailed description. Therefore, the true scope of technical protection of the present invention should be determined by the technical ideas of the attached claims. In addition, the present invention should be understood to include the spirit of the present invention as defined by the attached claims and all modifications, equals, and substitutes within the scope thereof.

1. A composition comprising:
a phosphodiesterase 5 inhibitor; and
a glucocorticoid receptor (GR) antagonist as active ingredients.

2. The composition of claim 1, wherein the phosphodiesterase 5 inhibitor is selected from the group consisting of mirodenafil, sildenafil, vardenafil, tadalafil, udenafil, dasantafil, avanafil, pharmaceutically acceptable salts, solvates, hydrates, and a mixture thereof.

3. The composition of claim 1, wherein the GR antagonist is CORT-108297, prednisolone, dexamethasone, mifepristone, ciclesonide, budesonide, cortisone, flunisolide; and at least one species selected from a group consisting of pharmaceutically acceptable salts, solvates, or hydrates thereof.

4. The composition for preventing and treating dementia of claim 1,
wherein
the phosphodiesterase 5 inhibitor is selected from the group consisting of mirodenafil, pharmaceutically acceptable salts, solvates, hydrates and a mixture thereof; and
the GR antagonist is selected from among CORT-108297, prednisolone, dexamethasone, mifepristone, ciclesonide, budesonide, cortisone, flunisolide; and at least one species selected from a group consisting of pharmaceutically acceptable salts, solvates, or hydrates thereof.

5. The composition of claim 1, wherein the phosphodiesterase 5 inhibitor is mirodenafil.

6. A method for preventing or treating neuroinflammation comprising:
administrating an effective amount of a pharmaceutical composition comprising the composition of claim 1.

7. A method for prevention or inhibiting formation and/or accumulation of beta-amyloid comprising:
administrating an effective amount of a pharmaceutical composition comprising the composition of claim 1.

8. A method for preventing or treating a neurodegenerative disease comprising:
administrating an effective amount of a pharmaceutical composition comprising the composition of claim 1.

9. The method of claim 8, wherein the neurodegenerative disease is dementia, Parkinson's disease (PD), Dementia with Lewy body (DLB), Alzheimer's disease (AD), Huntington's disease (HD), Multiple sclerosis (MS), Vascular Dementia (VaD), Amyotrophic Lateral Sclerosis (ALS), frontotemporal dementia, or a mixed etiologies thereof.

10. A method for inhibition of Aβ Oligomer/Fibril formation by reduction of Aβ aggregation comprising:
administrating an effective amount of a pharmaceutical composition comprising the composition of claim 1.

11. A method for inhibition of β-Amyloidogenic processing by reduction of BACE-1 comprising:
administrating an effective amount of a pharmaceutical composition comprising the composition of claim 1.

12. A method for reduction of extracellular Aβ monomers, oligomers & Aβ Fibril/Plaque by increase of the cerebral blood flow comprising:
administrating an effective amount of a pharmaceutical composition comprising the composition of claim 1.

13. A method for suppression of neuronal cell death, promotion of neurogenesis, synaptogenesis and/or angiogenesis by activation of NO/cGMP/PKG/CREB Pathway comprising:
administrating an effective amount of a pharmaceutical composition comprising the composition of claim 1.

14. A method for restoration of synaptic plasticity (synaptic Plasticity) by activation of Wint Signaling by inhibition of DKK-1 comprising:
administrating an effective amount of a pharmaceutical composition comprising the composition of claim 1.

15. A method for inhibition of production of APP and reduction of Aβ accumulation by suppression of positive feedback loop for Aβ production comprising:
administrating an effective amount of a pharmaceutical composition comprising the composition of claim 1.

16. A method for inhibition of formation of Aβ Fibril/plaque by removal of intracellular toxic and soluble Aβ oligomers by activation of autophagy comprising:
administrating an effective amount of a pharmaceutical composition comprising the composition of claim 1.
